(19) United States
(12) Patent Application Publication
(10) Pub. No.: US 2011/0054358 A1
(43) Pub. Date: Mar. 3, 2011

(54) GAIT AND POSTURE ANALYSIS SYSTEM AND METHOD USING CORRECTED FOOT PRESSURE

(75) Inventors: Min Ho KIM, Daejeon (KR); Ho Youl JUNG, Daejeon (KR); Jae Won JANG, Daejeon (KR); Sa Kwang SONG, Daejeon (KR); Soo Jun PARK, Seoul (KR); Seon Hee PARK, Daejeon (KR)

(73) Assignee: Electronics and Telecommunications Research Institute, Daejeon (KR)

(21) Appl. No.: 12/842,396

(22) Filed: Jul. 23, 2010

(30) Foreign Application Priority Data
Sep. 1, 2009 (KR) 10-2009-0082218

(51) Int. Cl. A61B 5/103

(52) U.S. Cl. 600/592

(57) ABSTRACT

There is provided a gait/posture analysis method which analyzes a gait/posture of a walker wearing a shoe having one or more pressure sensors attached thereon. The gait/posture analysis method includes: measuring a base foot pressure, which is generated when the walker wears the shoe, at arbitrary time intervals; calculating a representative base foot pressure by using the plurality of base foot pressures measured at the arbitrary time intervals; correcting a foot pressure measured by the pressure sensors by using the representative base foot pressure; calculating a foot-pressure related value, which is to be used for analyzing the gait/posture of the walker, by using the corrected foot pressure; and analyzing the gait/posture of the walker by using the foot-pressure related value.

### CROSS-REFERENCE TO RELATED APPLICATIONS

This application claims the priority of Korean Patent Application No. 10-2009-0082218 filed on Sep. 1, 2009, in the Korean Intellectual Property Office, the disclosure of which is incorporated herein by reference.

### BACKGROUND OF THE INVENTION

1. Field of the Invention

The present invention relates to a gait/posture analysis system and method using a corrected foot pressure, and more particularly, to technology which measures foot pressure through one or more pressure sensors mounted in a shoe to analyze the gait/posture of a person wearing the shoe, corrects the measured foot pressure using a representative base foot pressure, and then analyzes the gait/posture of the shoe-wearing person such that gait/posture analysis may be performed with precision.

2. Description of the Related Art

As interest in walking for health care and maintenance has increased, research for analyzing a walker's gait/posture by analyzing walker's foot pressure has been actively conducted so as to provide a corrective walking program.

In particular, a gait/posture analysis method has been proposed, which analyzes the gait/posture of a walker using foot pressure measured by one or more pressure sensors attached to an insole of a shoe worn by the walker.

However, the gait/posture analysis method according to the related art does not consider a base pressure which is applied to the pressure sensor attached to the shoe, when the walker simply wears the shoe. Therefore, since an incorrect measure of foot pressure is obtained, the reliability of the analyzed result inevitably decreases.

### SUMMARY OF THE INVENTION

An aspect of the present invention provides a gait/posture analysis system and method using a corrected foot pressure, which measures foot pressure through one or more pressure sensors mounted in a shoe to analyze the gait/posture of a person wearing the shoe, corrects the measured foot pressure using a representative base foot pressure, and then analyzes the gait/posture of the shoe-wearing person such that gait/posture analysis may be performed with precision.

According to an aspect of the present invention, there is provided a gait/posture analysis method which analyzes a gait/posture of a walker wearing a shoe having one or more pressure sensors attached thereon. The gait/posture analysis method includes: measuring a base foot pressure, which is generated when the walker wears the shoe, at arbitrary time intervals; calculating a representative base foot pressure by using the plurality of base foot pressure measurements measured at the arbitrary time intervals; correcting a foot pressure measurement measured by the pressure sensors by using the representative base foot pressure; calculating a foot-pressure related value (for example, center of pressure or center of gravity), which is to be used for analyzing the gait/posture of the walker, by using the corrected foot pressure measurement; and performing a gait/posture analysis on the walker by using the foot-pressure related value.

In the calculating of the representative base foot pressure, the representative base foot pressure may be calculated by using any one of the mean, a non-deviated-value mean, and the median of the base foot pressures.

In the correcting of the foot pressure measurement, the measured foot pressure may be corrected by subtracting the representative foot pressure from the measured foot pressure.

The foot-pressure related value may include a center of pressure (COP) and a center of gravity (COG).

In performing the gait/posture analysis, a value measured by a motion sensor may be additionally used to analyze the walker's gait/posture.

The gait/posture analysis method may further include displaying the walker's gait/posture analysis result.

According to another aspect of the present invention, there is provided a gait/posture analysis system using one or more corrected foot pressures, including: a sensor-mounted shoe worn by a walker and measuring a walker's foot pressure so as to transmit the foot pressure measurement to the outside; and a gait/posture analysis terminal correcting the foot pressure measurement received from the sensor-mounted shoe by using representative base foot pressures and then analyzing a walker's gait/posture by using the corrected foot pressure measurements.

The sensor-mounted shoe may include: one or more pressure sensors attached across the entire insole of the sensor-mounted shoe and measuring the walker's foot pressure; a control module sampling the foot pressure measurements measured by the one or more pressure sensors; and a wireless communications module transmitting the sampled foot pressures to the gait/posture analysis terminal.

The pressure sensors may include a force sensing resistor (FSR) sensor, and a piezoelectric sensor, or etc.

The sensor-mounted shoe may further include one or more motion sensors measuring motion-related data which are used for analyzing the walker's gait/posture, in addition to the foot pressure of the walker.

The one or more motion sensors may include one or more of an acceleration sensor, a gyro sensor, and a barometric pressure sensor.

The representative base foot pressure may be calculated by using any one of the mean, a non-deviated-value mean, and the median of a plurality of base foot pressures measured at arbitrary time intervals.

The gait/posture analysis module may include: a wireless communications module receiving the foot pressure measurement from the sensor-mounted shoe; a foot pressure correction module correcting the foot pressure measurement received from the wireless communication module by using the representative base foot pressure; a gait/posture value calculation module calculating a foot-pressure related value, which is to be used for analyzing the walker's gait/posture, by using the corrected foot pressure; and a gait/posture analysis module analyzing the walker's gait/posture by using the foot-pressure related value.

The foot pressure correction module may correct the foot pressure measurement by subtracting the representative base foot pressure from the foot pressure measurement.

The foot-pressure related value may include a COP (Center Of Pressure) and a COG (Center Of Gravity).

The gait/posture analysis module may analyze the walker's gait/posture by additionally using a value measured by a motion sensor attached on the sensor-mounted shoe.

The gait/posture analysis terminal may further include a display module displaying the gait/posture analysis result obtained by the gait/posture analysis module.

The gait/posture analysis terminal may further include a database storing one or more of the representative base foot pressures, normal gait/posture data, and the gait/posture analysis result.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a flow chart explaining a gait/posture analysis method using a corrected foot pressure measurement according to an embodiment of the present invention; and

FIG. 2 is a configuration diagram of a gait/posture analysis system using a corrected foot pressure measurement according to another embodiment of the present invention.

### DETAILED DESCRIPTION OF THE PREFERRED EMBODIMENT

Exemplary embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. Like reference numerals in the drawings denote like elements, and thus their description will be omitted.

When it is described that one component is ‘connected’ to another component, it includes a case in which the one component is ‘indirectly connected’ to another component with a different component interposed therebetween as well as a case in which the one component is ‘directly connected’ to another component. Furthermore, when it is described that one component ‘includes’ another component, it means that the one component does not exclude a different component, but may include the different component.

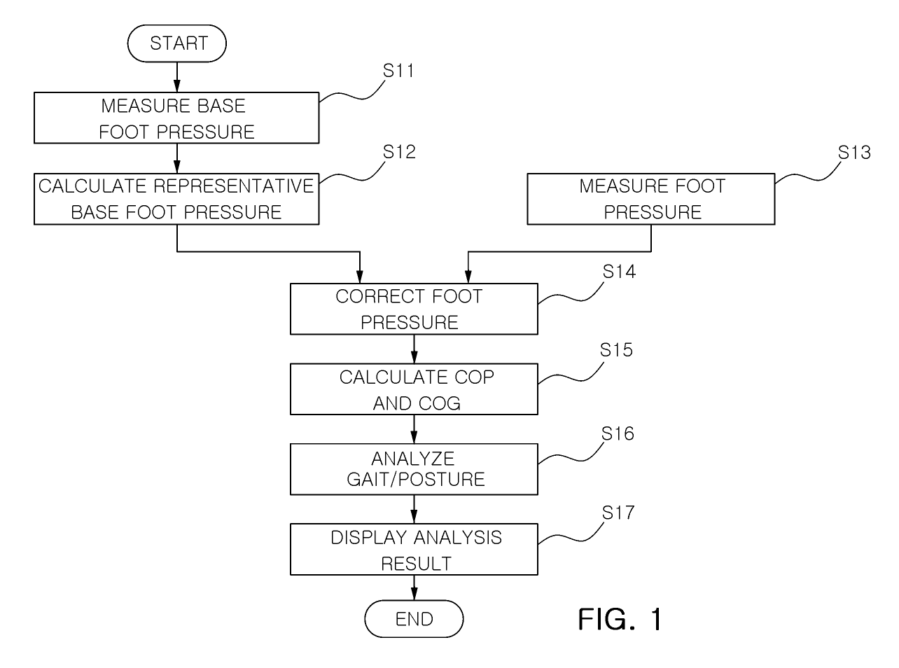

FIG. 1 is a flow chart explaining a gait/posture analysis method using a corrected foot pressure measurement according to an embodiment of the present invention.

According to the embodiment of the present invention, one or more pressure sensors are attached on an insole of a shoe worn by a walker. Furthermore, when the gait/posture of the walker is analyzed by using foot pressures measured by the pressure sensors, a corrected foot pressure for more precise gait/posture analysis is used to perform the gait/posture analysis.

First, when the walker wears the shoe, the pressure sensors attached on the shoe measures the walker's base foot pressure (S11). Base foot pressure refers to a walker's foot pressure which is measured by the pressure sensor attached to the shoe, when the walker wears the shoe and then holds the foot in the air, that is, in a state in which foot pressure generated by walking or standing is not applied.

Then, a representative base foot pressure is calculated by using the measured base foot pressures (S12). More specifically, representative base foot pressure is calculated by using the plurality of base foot pressures measured at arbitrary time intervals. The representative base foot pressure may be calculated by using the mean, a non-deviated-value mean, and the median of the base foot pressures. The non-deviated-value mean refers to a mean value calculated by using the base foot pressures excluding the base foot pressures deviating from a predetermined reliable range (for example, 95% or 99% confidence intervals), among the plurality of base foot pressures. When the non-deviated-value mean is used, it is possible to prevent the mean value from being distorted by the bouncing values.

Meanwhile, the pressure sensors attached to the shoe worn by the walker measure foot pressures when the walker walks or stands erect (S13).

The representative base foot pressure calculated in such a manner is used to correct the foot pressure measurements measured by the pressure sensors attached to the shoe worn by the walker (S14). For example, the correction of the foot pressure measurement may be performed by subtracting the representative foot pressure from the measured foot pressure measurement. At this time, a negative number which may occur in the subtraction is replaced with zero.

Then, foot-pressure related values including a center of pressure (COP) and a center of gravity (COG) are calculated by using the corrected foot pressures (S15). The foot-pressure related values are used for the gait/posture analysis.

In the case of the COP, a maximum pressure value or

$${\sum }_{i} {Pr}_{i} \cdot {Pt}_{i}$$

may be calculated by using the corrected foot pressures. At this time,

$${Pr}_{i} = \frac{{P}_{i}}{{\sum }_{i} {P}_{i}} ,$$

where Ptᵢ represents two-dimensional coordinates (x, y) of a pressure point i, and Pᵢ represents a corrected foot pressure at the pressure point i. The two-dimensional coordinates of the pressure point i may be obtained by using a mapping table which converts IDs or relative coordinates of the respective foot pressure points into absolute coordinates.

Subsequently, the foot-pressure related values such as the COP and the COG are used to analyze gait/postures including the walking gait/posture and stationary gait/posture of the walker (S16). In this case, a plurality of values measured by various motion sensors including an acceleration sensor, a gyro sensor, and a barometric pressure sensor may be used to perform a gait/posture analysis with greater precision, in addition to the foot-pressure related values. Furthermore, the values acquired by analyzing gait/postures through the above-described process may be compared with predetermined normal gait/posture data to determine when the gait/posture of the walker is in a normal state or not.

Then, the gait/posture analysis result is displayed (S17) so that the walker can understand and correct his or her bad gait/posture habit. For example, the result of the stationary gait/posture analysis may inform the walker that his or her body is supported on one leg, is bent forward slightly or that his or her chest is pushed forward excessively. Therefore, the walker may understand and correct his or her bad gait/posture habit.

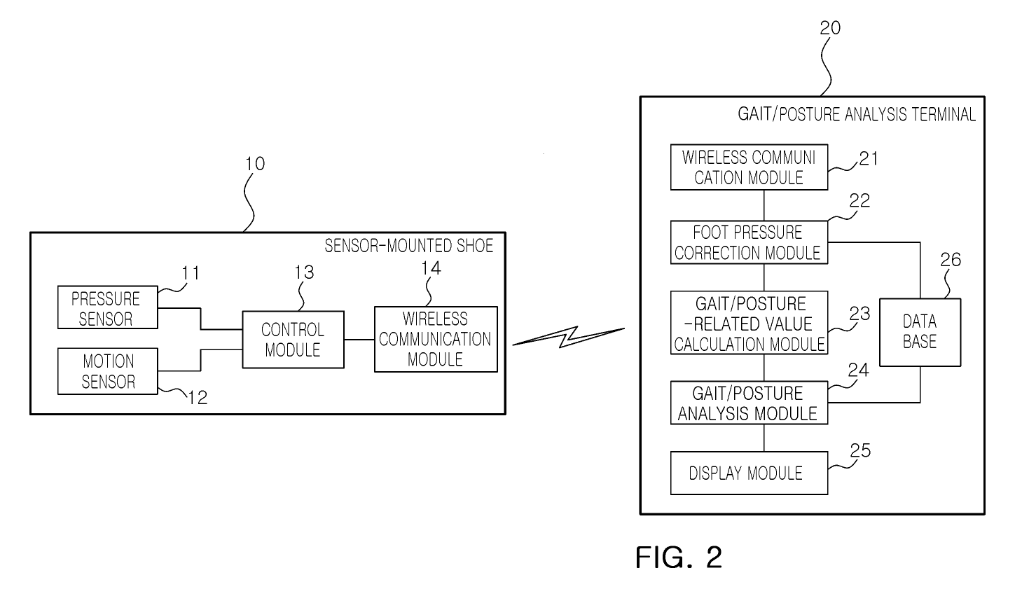

FIG. 2 is a configuration diagram of a gait/posture analysis system using a corrected foot pressure measurement according to another embodiment of the present invention.

The gait/posture analysis system according to the embodiment of the present invention may include a sensor-mounted shoe 10 and a gait/posture analysis terminal 20.

The sensor-mounted shoe 10, which measures a walker's foot pressures while the walker is wearing the sensor-mounted shoe 10 so as to transmit measurements to the gait/posture analysis terminal 20 performing gait/posture analysis, includes one or more pressure sensors 11, a control module 13, and a wireless communication module 14. The sensor-mounted shoe 10 may further include one or more motion sensors 12.

The pressure sensors 11 serve to measure the walker's foot pressure measurements, and may include force sensing resistor (FSR) sensors, piezoelectric sensors, or etc. Furthermore, one or more pressure sensors 11 may be arranged across the entire insole of the shoe such that the entire foot of the walker is covered.

The motion sensors 12 serve to measure acceleration, angular velocity, atmospheric pressure and so on, which may be used for the gait/posture analysis of the walker, in addition to the walker's foot pressure measurements. The motion sensors 12 may include an acceleration sensor, a gyro sensor, and a barometric pressure sensor.

The control module 13 serves to perform primary processing on data sensed by the pressure sensors 11 and the motion sensors 12. For example, the control module 13 may sample the sensed data. The control module 13 may include a micro controller unit (MCU).

The wireless communication module 14 serves to transmit the data sampled by the control module 13 to the gait/posture analysis terminal 20, and may be implemented by wireless communication technology such as ZigBee or Bluetooth.

The gait/posture analysis terminal 20 serves to analyze the gait/posture of the walker by analyzing the data (foot pressure) received from the sensor-mounted shoe 10. The gait/posture analysis terminal 20 corrects the foot pressure measurements measured during walking or standing without using the foot pressure as it is, and then performs the gait/posture analysis using the corrected foot pressure. The gait/posture analysis terminal 20 includes a wireless communication module 21, a foot pressure correction module 22, a gait/posture-related value calculation module 23, a gait/posture analysis module 24, a display module 25, and a database 26.

The wireless communications module 21 serves to receive the measured data from the sensor-mounted shoe 10, and may be implemented by using the same communications technology as that of the wireless communications module 14 mounted on the sensor-mounted shoe 10, that is, the wireless communication technology such as ZigBee or Bluetooth.

The foot pressure correction module 22 corrects the measured data received from the sensor-mounted shoe 10 through the wireless communication module 21, that is, the measured foot pressure by using a pre-calculated representative base foot pressure. For example, the foot pressure correction module 22 may calculate a corrected foot pressure by subtracting the representative base foot pressure from the measured foot pressure measurement. The method for calculating the representative base foot pressure has been described with reference to FIG. 1. Therefore, the detailed descriptions thereof will be omitted.

The gait/posture-related value calculation module 23 serves to calculate foot-pressure related values such as the COP and the COG, which are used for gait/posture analysis, by using the foot pressure corrected by the foot pressure correction module 22.

The gait/posture analysis module 24 serves to analyze gait/postures including the gait pattern and the stationary posture of the walker (shoe wearer) by using the foot-pressure related values calculated by the gait/posture-related value calculation module 23. Furthermore, when the measured data received through the wireless communication module 21 include values measured by the motion sensors 12, the gait/posture analysis module 24 may analyze the gait/posture of the walker more precisely by using the foot-pressure related values and the values measured by the motion sensors 12. Furthermore, the gait/posture analysis module 24 may compare the analyzed data with normal gait/posture data stored in the data base 26 to determine whether the gait/posture of the walker is normal or not.

The display module 25 displays gait/posture-related information including the walker's gait/posture analysis result and information for gait/posture correction which is related to bad gait/posture.

The database 26 serves to store data required for analyzing the walker's gait/posture and the gait/posture analysis result data. More specifically, the database 26 may store all kinds of information related to the gait/posture analysis, including the plurality of base foot pressures measured at arbitrary intervals, the representative base foot pressure calculated from the plurality of base foot pressures, the normal gait/posture data, the information for gait/posture correction, and the gait/posture analysis result.

According to the embodiments of the present invention, the foot pressure measurement may be corrected by using the representative base foot pressure and then used to analyze the gait/posture of the shoe-wearing walker. Therefore, it is possible to perform the gait/posture analysis with greater precision.

While the present invention has been shown and described in connection with the exemplary embodiments, it will be apparent to those skilled in the art that modifications and variations can be made without departing from the spirit and scope of the invention as defined by the appended claims.

What is claimed is:

1. A gait/posture analysis method which analyzes a gait/posture of a walker wearing a shoe having one or more pressure sensors attached thereon, the gait/posture analysis method comprising:
measuring a base foot pressure, which is generated when the walker wears the shoe, at arbitrary time intervals;
calculating a representative base foot pressure by using the plurality of base foot pressure measurements measured at the arbitrary time intervals;
correcting a foot pressure measurement measured by the pressure sensors by using the representative base foot pressure;
calculating a foot-pressure related value, which is to be used for analyzing the gait/posture of the walker, by using the corrected foot pressure measurement; and
performing a gait/posture analysis on the walker by using the foot-pressure related value.

2. The gait/posture analysis method of claim 1, wherein, in the calculating of the representative base foot pressure, the representative base foot pressure is calculated by using any one of the mean, a non-deviated-value mean, and the median of the base foot pressures.

3. The gait/posture analysis method of claim 1, wherein, in the correcting of the foot pressure measurement, the measured foot pressure is corrected by subtracting the representative foot pressure from the measured foot pressure.

4. The gait/posture analysis method of claim 1, wherein the foot-pressure related value comprises a center of pressure (COP) or a center of gravity (COG).

5. The gait/posture analysis method of claim 1, wherein, in the performing the gait/posture analysis, a value measured by a motion sensor is additionally used to analyze the walker's gait/posture.

6. The gait/posture analysis method of claim 1, further comprising displaying the walker's gait/posture analysis result.

7. A gait/posture analysis system using a corrected foot pressure comprising:
a sensor-mounted shoe worn by a walker and measuring a walker's foot pressure so as to transmit the foot pressure measurement to the outside; and
a gait/posture analysis terminal correcting the foot pressure measurement received from the sensor-mounted shoe by using a representative base foot pressure and then analyzing a walker's gait/posture by using the corrected foot pressure measurement.

8. The gait/posture analysis system of claim 7, wherein the sensor-mounted shoe comprises:
one or more pressure sensors attached across the entire insole of the sensor-mounted shoe and measuring the walker's foot pressure;
a control module sampling the foot pressure measurements measured by the one or more pressure sensors; and
a wireless communications module transmitting the sampled foot pressures to the gait/posture analysis terminal.

9. The gait/posture analysis system of claim 8, wherein the pressure sensors comprise a force sensing resistor (FSR) sensor or a piezoelectric sensor.

10. The gait/posture analysis system of claim 8, wherein the sensor-mounted shoe further comprises one or more motion sensors measuring motion-related data which are used for analyzing the walker's gait/posture, in addition to the foot pressure of the walker.

11. The gait/posture analysis system of claim 10, wherein the one or more motion sensors comprise one or more of an acceleration sensor, a gyro sensor, or a barometric pressure sensor.

12. The gait/posture analysis system of claim 7, wherein the representative base foot pressure is calculated by using any one of the mean, a non-deviated-value mean, and the median of a plurality of base foot pressures measured at arbitrary time intervals.

13. The gait/posture analysis system of claim 7, wherein the gait/posture analysis terminal comprises:
a wireless communications module receiving the foot pressure measurement from the sensor-mounted shoe;
a foot pressure correction module correcting the foot pressure measurement received from the wireless communication module by using the representative base foot pressure;
a gait/posture-related value calculation module calculating a foot-pressure related value, which is to be used for analyzing the walker's gait/posture, by using the corrected foot pressure; and
a gait/posture analysis module analyzing the walker's gait/posture by using the foot-pressure related value.

14. The gait/posture analysis system of claim 13, wherein the foot pressure correction module corrects the foot pressure measurement by subtracting the representative base foot pressure from the foot pressure measurement.

15. The gait/posture analysis system of claim 13, wherein the foot-pressure related value comprises a COP or a COG.

16. The gait/posture analysis system of claim 13, wherein the gait/posture analysis module analyzes the walker's gait/posture by additionally using a value measured by a motion sensor attached on the sensor-mounted shoe.

17. The gait/posture analysis system of claim 13, wherein the gait/posture analysis terminal further comprises a display module displaying the gait/posture analysis result obtained by the gait/posture analysis module.

18. The gait/posture analysis system of claim 13, wherein the gait/posture analysis terminal further comprises a database storing one or more of the representative base foot pressure, normal gait/posture data, and the gait/posture analysis result.
